(19) United States
(12) Patent Application Publication
(10) Pub. No.: US 2026/0217636 A1
(43) Pub. Date: Jul. 30, 2026

(54) PROCESS FOR THE SYNTHESIS OF ISOPHORONE IN THE LIQUID PHASE WITH RECYCLING OF THE ALKALINE CATALYST BY ELECTRODIALYSIS

(71) Applicant: ARKEMA FRANCE, Puteaux (FR)

(72) Inventors: Christophe RUPPIN, La Chambre (FR); Claudine BOISSON, Singapore (SG)

(73) Assignee: ARKEMA FRANCE, Puteaux (FR)

(21) Appl. No.: 19/139,846

(22) PCT Filed: Dec. 18, 2023
(86) PCT No.: PCT/EP2023/086481

§ 371 Date: Jun. 16, 2025

(30) Foreign Application Priority Data
Dec. 19, 2022 (FR) FR2213789

(51) Int. Cl. C07C 45/75; C07C 45/82

(52) U.S. Cl. CPC C07C 45/75; C07C 45/82; C07C 2601/16

(57) ABSTRACT

The present invention relates to a process for the liquid-phase synthesis of isophorone by alkaline self-condensation of acetone, comprising the following successive steps:
a) condensation reaction of acetone within a reactor in an alkaline medium, followed by
b) distillation, optionally reactive distillation, of the reaction mixture obtained from the reactor, followed by
c) separation of the stream recovered at the bottom of the distillation column, optionally reactive distillation column, of step b) so as to separate the alkaline aqueous phase from the organic phase, followed by
d) extraction and/or purification of the organic phase to recover isophorone,
characterized in that the method comprises the following successive steps:
e) electrodialysis treatment of the alkaline aqueous phase recovered at the end of step c)
f) recycling to the reactor in step a) of the aqueous phase obtained from the electrodialysis which has an alkali metal hydroxide content greater than the alkali metal hydroxide content of the aqueous phase recovered at the end of step c).

### TECHNICAL FIELD

The present invention relates to a process for the liquid-phase synthesis of isophorone by alkaline self-condensation of acetone, comprising the electrodialysis treatment of an aqueous effluent generated during the synthesis.

### TECHNICAL BACKGROUND

Isophorone (or 3,5,5-trimethylcyclohex-2-enone) is an α,β-unsaturated cyclic ketone of increasing use as a synthetic intermediate, in particular for the manufacture of isophorone diamine, which is used as a hardener for epoxy resins, isophorone diisocyanate, which is used as a polyurethane monomer, 3,5-xylenol, which is used as a precursor of PCMX (antimicrobial agent), keto-isophorone, which is a synthesis intermediate of vitamin E and 3,5,5-trimethylcyclohexanol, which is used as a precursor of homosalate (UV absorber). Isophorone is also an excellent high-boiling-point solvent for many natural and synthetic resins. It is also used as a solvent in the paint, ink and varnish industries, and also in agrochemistry for the formulation of emulsifiable pesticide concentrates.

Isophorone is conventionally obtained by catalytic self-condensation of 3 molecules of acetone, according to the following reaction:

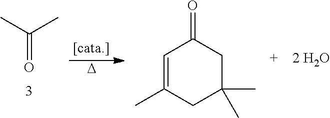

The reaction is carried out in the liquid phase or in the gas phase.

The gas-phase processes described in the literature essentially use solid heterogeneous catalysts, whereas the liquid-phase processes use homogeneous or heterogeneous catalytic systems.

The synthesis of isophorone by condensation of acetone in the liquid phase is carried out almost exclusively under alkaline conditions at high temperature and under high pressure; the alkaline catalysis being carried out most often by using an aqueous solution of sodium hydroxide or potassium hydroxide.

Owing to the low solubility of the mineral base in acetone, processes that aim to promote the contact of acetone with the catalyst are sought. Thus, it is known from document U.S. Pat. No. 2,344,226 to carry out the synthesis in a stirred reactor. Document FR 1 238 954 discloses a synthesis using a tubular reactor with internal packing. Document U.S. Pat. No. 2,399,976 discloses a synthesis using a tubular reactor equipped with a recirculation system. Documents CN 102367223 and CN 102516051 disclose a synthesis using a premixing system, such as a static mixer. It is also known from document FR 1042057 to replace the alkaline aqueous solution with an alkaline alcoholic solution.

The synthesis can be carried out continuously in a tubular reactor without special mixing equipment via the use of very low concentrations by weight of sodium hydroxide or potassium hydroxide. Generally, the catalyst concentrations are less than 1% by weight, or even of the order of 0.1% by weight relative to the total weight of the reaction mixture. This low concentration allows a single-phase mixture. These processes are described in the documents FR 1 316 515, DD145096, EP 2 649 032, EP 2 707 352 and EP 2 837 618.

The synthesis can also be carried out by reactive distillation via the injection of acetone and an aqueous solution of sodium hydroxide or potassium hydroxide into a reactive distillation column so as to maintain a low concentration of sodium hydroxide or potassium hydroxide (<0.1% by weight relative to the total weight of the reaction mixture) and to react acetone countercurrent to the sodium hydroxide or potassium hydroxide. This process is described in documents FR 1 315 788, FR 2 271 191 and FR 2 328 686.

The acetone self-condensation reaction and/or the hydrolysis of the reaction intermediates and polycondensation by-products generates an alkaline aqueous effluent. In addition to the initial alkaline aqueous solution used, this effluent comprises the water resulting from the self-condensation reaction of acetone to isophorone and contains small amounts of organic products (mainly isophorone). This effluent must undergo an expensive specific treatment to limit its environmental impact when discharged.

A process is known from document U.S. Pat. No. 8,889,914 B2 in which the aqueous phase of the hydrolysis column is treated by distillation and flash evaporation, in order to recycle a portion of the organic compounds and water contained in this stream. This process makes it possible to recover most of the organics and water, but not the catalyst. Thus, all of the catalyst injected into the reaction is lost. The problem of managing the residual aqueous effluent still arises: it must be neutralized, then the salts resulting from this neutralization must be removed before the water is discharged into the natural environment.

There is therefore a need for a process for the synthesis of isophorone that is more economical in terms of reagent, more economical in terms of energy and more environmentally friendly. The desired process must generate less waste, without however losing selectivity or productivity.

### BRIEF DESCRIPTION OF THE INVENTION

The present invention relates to a process, preferably a continuous process for the liquid-phase synthesis of isophorone by alkaline self-condensation of acetone, comprising the following successive steps:
a) condensation reaction of acetone within a reactor in an alkaline medium, followed by
b) distillation, optionally reactive distillation, of the reaction mixture obtained from the reactor, followed by
c) separation of the stream recovered at the bottom of the distillation column, optionally reactive distillation column, of step b) so as to separate the alkaline aqueous phase from the organic phase, followed by
d) extraction and/or purification of the organic phase to recover isophorone,
characterized in that the method comprises the following successive steps:
e) continuous or batch electrodialysis treatment of the alkaline aqueous phase recovered at the end of step c)
f) recycling to the reactor in step a) of the aqueous phase obtained from the electrodialysis which has an alkali metal hydroxide content greater than the alkali metal hydroxide content of the aqueous phase recovered at the end of step c).

Other advantageous characteristics of the process according the invention are specified below:
the electrodialysis apparatus comprises at least one ion-exchange membrane comprising a polymer-based matrix comprising at least one fluorinated polymer or copolymer, preferably PVDF,
the electrodialysis apparatus has a total active exchange surface area of between 1 and 10 m² per tonne of alkaline aqueous phase to be treated, and preferably of between 2 and 5 m² per tonne of alkaline aqueous phase to be treated,
a current density of between 20 and 200 mA/cm², and preferably of between 30 and 100 mA/cm², is applied to the electrodialysis apparatus,
the electrodialysis apparatus includes several electrodialysis units in parallel or in series,
the aqueous alkali metal hydroxide solution is an aqueous solution of sodium hydroxide or potassium hydroxide,
the concentration of alkali metal hydroxide in the aqueous phase present within the reactor is greater than or equal to 50 g/l, preferably between 50 and 200 g/l, preferably between 80 and 150 g/l and more preferentially between 100 and 150 g/l,
the ratio of the mass flow rate of the stream of aqueous alkali metal hydroxide solution supplied (Qalkali metal hydroxide) to the mass flow rate of the organic stream supplied (Qorga) is between 0.25 and 1.0, preferably between 0.4 and 0.8 and more preferentially between 0.5 and 0.7,
the concentration of alkali metal hydroxide in the aqueous solution at the feed zone is between 5 and 40 g/l, preferably between 10 and 40 g/l and more preferentially between 15 and 35 g/l.

The invention also relates to the use of an electrodialysis apparatus as defined above, for treating at least one alkaline aqueous effluent obtained from a process for the synthesis of isophorone by alkaline self-condensation of acetone in the liquid phase.

### BRIEF DESCRIPTION OF THE FIGURE

FIG. 1 is a scheme of the device performing the claimed process.

### DETAILED DESCRIPTION

Other characteristics, aspects, subjects and advantages of the present invention will emerge even more clearly on reading the description that follows.

It is specified that the expressions “from . . . to . . . ” and “between . . . and . . . ” used in the present description should be understood as including each of the limits mentioned.

The process according to the invention comprises the following consecutive steps:

Step a) Condensation Reaction of Acetone within a Reactor in an Alkaline Medium

The synthesis can be carried out by injection, preferably continuous injection, of a stream of an aqueous alkali metal hydroxide solution and a stream of an organic solution comprising acetone through a reactor R.

Advantageously, the ratio of the mass flow rate of the stream of aqueous alkali metal hydroxide solution supplied (Qalkali metal hydroxide) to the mass flow rate of the organic stream supplied (Qorga) is between 0.25 and 1.0, preferably between 0.4 and 0.8 and more preferentially between 0.5 and 0.7.

The concentration of alkali metal hydroxide in the aqueous solution at the feed zone may be between 5 and 40 g/l, preferably between 10 and 40 g/l and more preferentially between 15 and 35 g/l.

The streams can be preheated beforehand using heat exchangers.

The reaction temperature within the reactor may be between 180° C. and 250° C., and preferably between 200° C. and 230° C., and under an absolute pressure of between 30 and 50 bar, preferably between 35 and 45 bar, and even more preferentially between 38 and 42 bar.

The reactor R is preferably a tubular reactor, and more particularly a tubular reactor in a vertical position. Furthermore, it may, where appropriate, consist of several tubular reactors fed in parallel.

Preferably, the concentration of alkali metal hydroxide in the aqueous phase present within the reactor is greater than or equal to 50 g/l, preferably between 50 and 200 g/l, preferably between 80 and 150 g/l and more preferentially between 100 and 150 g/l.

The reaction mixture is recovered at the outlet of the reactor and conveyed to a distillation column.

### Step b): Distillation

The reaction mixture recovered at the reactor outlet is distilled through a column, optionally a reactive column.

According to one embodiment of the process according to the invention, the process comprises a hydrolysis reactive distillation, making it possible to hydrolyse the heavy products. According to this eventuality, the process may comprise two or three successive distillations, so as to purify, at each distillation, the fraction containing predominantly isophorone.

According to another embodiment of the process according to the invention, the process does not comprise reactive distillation, but a succession of non-reactive distillations. Preferably, the process comprises four to six successive distillations, so as to purify, at each distillation, the fraction containing predominantly isophorone. The heavy products isolated by means of these distillations can be recycled.

At the end of the first reactive or non-reactive distillation, the unconverted acetone is recovered at the top of the column and the concentrated crude reaction mixture is drawn off at the bottom of the column. The acetone recovered at the top of the column can be recycled to the reactor R.

### Step c): Separation

The concentrated crude reaction mixture drawn off at the bottom of the first distillation column is separated, preferably by decantation. The alkaline aqueous phase can be separated from the isophorone-rich organic phase by means of a decanter.

### Optional Neutralization of the Organic Phase

Any alkali metal hydroxide present in the isophorone-rich organic phase recovered in the separation step c) may be neutralized. This neutralization may be carried out by any technique known to those skilled in the art, but preferably by means of a mineral acid providing a buffer effect. Preferably, phosphoric acid is used.

Step d): Extraction and/or Purification

The isophorone-rich organic phase recovered in the separation step, and then optionally neutralized, is purified. Preferably it is distilled, preferably under reduced pressure, in order to extract the polycondensation by-products predominantly at the bottom of the column and to recover a stream consisting predominantly of isophorone at the top of the column.

### Subsequent Distillation(s)

The stream consisting predominantly of isophorone recovered in the preceding step may undergo several successive distillations so as to obtain a high degree of purification.

### Step e): Electrodialysis Treatment

The aqueous stream leaving separation step c), preferably leaving a decanter, is subjected to an electrodialysis treatment.

This treatment step can be carried out batchwise or continuously.

This alkaline aqueous phase is treated by electrodialysis in order to recover:
an aqueous phase having an alkali metal hydroxide content greater than the alkali metal hydroxide content of the aqueous phase recovered at the end of step c); and
an aqueous phase having an alkali metal hydroxide content lower than the alkali metal hydroxide content of the aqueous phase recovered at the end of step c).

In other words, the electrodialysis apparatus makes it possible to obtain a catalyst-enriched phase and a catalyst-depleted phase.

The electrodialysis is carried out in any equipment known to those skilled in the art allowing the migration of ions through selective (anionic or cationic) ion-exchange membranes under the action of an electric field applied perpendicularly to the membranes. The electrodialysis apparatus comprises at least one electrodialysis unit comprising at least 2 electrodes: an anode and a cathode, and anionic and cationic membranes arranged alternately in parallel so as to constitute at least one concentration compartment and at least one dilution compartment. Advantageously, the electrodialysis apparatus consists of several electrodialysis units in parallel or in series.

In the case of the alkaline aqueous phase loaded with alkali metal hydroxide, the alkali metal cations pass through the cationic membranes, denoted MEC below, and the OH⁻ anions pass through the anionic membranes, denoted MEA below. Thus, in the case of the alkaline aqueous phase loaded with sodium hydroxide, the Na⁺ cations pass through the cationic membranes (MEC), and the OH-anions pass through the anionic membranes (MEA). In the case of the alkaline aqueous phase loaded with potassium hydroxide, the K+ cations pass through the cationic membranes (MEC), and the OH⁻ anions pass through the anionic membranes (MEA). These ion-exchange membranes comprise a polymer matrix, grafted onto which are functional groups, preferentially of the sulfonic —(SO₃)⁻ or phosphoric —(PO₃)²⁻ type for the MECs and preferably of the alkylammonium —(NR₃)⁺, —(NHR₂)⁺, —(NH₂R)⁺ or alkylsulfonium —(SR₂)⁺ type for the MEAs; the R group(s), which may be identical or different, denote(s) a saturated C₁-C₆ alkyl group.

Preferably, the ion-exchange membranes comprise polymer-based matrices which may in particular comprise fluorinated polymers or copolymers, including in particular PVDF.

Preferably, the total active exchange surface area constituted by all of the ion-exchange membranes of the electrodialysis apparatus is between 1 and 10 m² per tonne of alkaline aqueous phase to be treated, and preferably of between 2 and 5 m² per tonne of alkaline aqueous phase to be treated.

Preferably, a current density of between 20 and 200 mA/cm², and in particular of between 30 and 100 mA/cm², is applied to the electrodialysis apparatus.

According to one embodiment of the process according to the invention, the electrodialysis apparatus has a total active exchange surface area of between 1 and 10 m² per tonne of alkaline aqueous phase to be treated and a current density of between 20 and 200 mA/cm².

According to one preferred embodiment of the process according to the invention, the electrodialysis apparatus has a total active exchange surface area of between 2 and 5 m² per tonne of alkaline aqueous phase to be treated and a current density of between 30 and 100 mA/cm² is applied to the electrodialysis apparatus.

The electrodialysis treatment of the aqueous effluents makes it possible to recycle the catalyst and thus avoid its loss in the wastewater, and consequently enables the treatment of this wastewater. This treatment also makes it possible to remove excess water from the process.

This excess amount of water corresponds to the water formed in the reactor minus the water consumed in the hydrolysis column, minus the water dissolved in the crude isophorone which is removed at the top of one of the subsequent distillation columns.

The catalyst-depleted aqueous stream at the outlet of the electrodialysis can be sent to a wastewater treatment plant S_{E}.

### Step f): Recycling

The catalyst-enriched aqueous phase obtained from the electrodialysis apparatus, i.e. having an alkali metal hydroxide content greater than the alkali metal hydroxide content of the aqueous phase recovered at the end of step c), is recycled to the reaction step.

### Use

The invention also relates to the use of an electrodialysis apparatus as defined above, for treating at least one alkaline aqueous effluent obtained from a process for the synthesis of isophorone by alkaline self-condensation of acetone in the liquid phase. The aqueous phase enriched in alkaline catalyst obtained can be recycled to the reactor of the acetone self-condensation reaction.

For the purposes of the present invention, the term “aqueous effluent” is understood to mean any alkaline aqueous solution produced by the isophorone synthesis process. Preferably, the synthesis process is as defined above, that is to say that it comprises steps a) to d) defined above.

The invention also relates to a process for the treatment of at least one alkaline aqueous effluent as defined above obtained from a process for the synthesis of isophorone by alkaline self-condensation of acetone in the liquid phase as defined above, comprising a step of treatment by electrodialysis apparatus as defined above.

### DESCRIPTION OF THE FIGURE

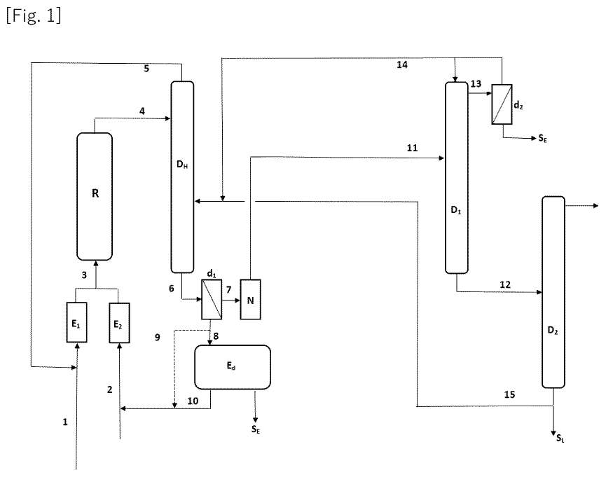

FIG. 1 represents one embodiment of the process according to the invention.

Acetone is introduced via line 1 into heat exchanger E1. The aqueous alkali metal hydroxide solution is introduced via line 2 into heat exchanger E2. The preheated streams are recovered in a line 3 and introduced into the tubular reactor R.

The reaction mixture obtained from the tubular reactor R is introduced into the hydrolysis reactive distillation column D_{H} via line 4. Hydrolysis of the reaction mixture is carried out under reduced pressure.

The acetone which has not been converted in the tubular reactor R is recovered at the top of column D_{H}. This fraction is recycled via the line 5 to the line 1.

The concentrated crude reaction mixture is recovered at the bottom of column D_{H} and is conveyed to the decanter d1 via line 6.

The decanter d1 separates the aqueous phase from the organic phase.

The alkaline aqueous phase is removed via the line 8. This line 8 brings all or part of the aqueous phase to the electrodialysis apparatus E_{d}. Any remaining aqueous phase, which is not brought to the electrodialysis apparatus, is recycled as is to the reaction step via the line 9 to the line 10.

The alkaline aqueous phase is treated by electrodialysis within E_{d} in order to:
recover an aqueous phase enriched in catalyst (alkali metal hydroxide), recycled to the reaction step via line 10, and
remove an aqueous phase depleted in catalyst, sent to a wastewater treatment S_{E}.

The organic phase obtained from the decanter d1 is conveyed to the neutralizer N via the line 7. The neutralized organic phase is conveyed to the distillation column D1 via line 11.

The distillation under reduced pressure using the distillation column D1 makes it possible to recover the residual water and any light organic impurities at the top of column D1 and, at the bottom of the column, a stream comprising predominantly isophorone which is transferred via the line 12 to the distillation column D2.

The stream extracted at the top of column D1 is conveyed via a line 13 to a decanter d2 in order to separate an aqueous phase sent to a wastewater treatment S_{E} and an organic phase sent back to the reflux of column D1 and, where appropriate, partly recycled to the hydrolysis column D_{H} via line 14 to line 15.

The distillation under reduced pressure using distillation column D2 allows isophorone with a purity greater than 99% to be recovered at the top of column D2 and C₃ₙH₍₄ₙ₊₂₎O polycondensation by-products to be recovered at the bottom of column. These by-products are recycled, completely or in part, via the line 15 to the reactive column D_{H}. The portion of the recovered fraction containing the C₃ₙH₍₄ₙ₊₂₎O polycondensation by-products, which is not recycled, is recovered (S_{L}).

Preferably, the concentration by weight of isophorone in the stream at the feed of D1 is greater than 70%, preferably greater than 75%.

Preferably, the concentration by weight of isophorone in the stream at the feed of D2 is greater than 75%, preferably greater than 80%.

The examples that follow illustrate the present invention but are not in any way limiting.

### EXAMPLES

These examples illustrate the implementation of the recovery of the catalyst on the basis of a unit production of pure isophorone of 1 t/h, using sodium hydroxide as catalyst.

The flow rate of the stream of alkaline aqueous phase at the outlet of the decanter d₁ at the bottom of the hydrolysis column D_{H} is 4.67 t/h and the concentrations by weight of sodium hydroxide (catalyst) and of isophorone in this aqueous phase are 2.8% and 0.85% respectively.

### Ex.1 (Comparative, Outside the Invention): Without any Recycling of the Aqueous Phase

By removing all of the alkaline aqueous phase to a wastewater treatment, the consumption at the feed of the reaction step of the isophorone synthesis process is 131 kg sodium hydroxide and 4.3 m³ water per tonne of pure isophorone produced, and the loss of isophorone contained in the alkaline aqueous phase is 39.7 kg per tonne of pure isophorone.

### Ex.2 (Comparative, Outside the Invention): With Partial Recycling of the Alkaline Aqueous Phase but without Concentration Treatment of the Purge

After having carried out a purge of 0.24 t/h on the stream of alkaline aqueous phase in order to remove the excess water generated by the reaction, the majority remaining portion of the stream, i.e. 4.43 t/h, is recycled to the reaction step and the purge is removed as is to a wastewater treatment.

The consumption at the feed of the reaction step of the isophorone synthesis process is then 7 kg of sodium hydroxide (also taking into account the loss of NaOH dissolved in the crude isophorone stream and representing around 0.25 kg per tonne of pure isophorone). As the make-up sodium hydroxide is added in the form of an aqueous solution of sodium hydroxide with a concentration by weight of 30.5%, the water consumption is then 16 litres of water per tonne of pure isophorone. And the loss of isophorone contained in the alkaline aqueous phase purge represents 2.0 kg per tonne of pure isophorone.

### Ex.3 (in Accordance with the Invention)

The 4.67 t/h stream of alkaline aqueous phase leaving the decanter d1 is treated in an electrodialysis apparatus supplied with a constant direct current of 60 mA/cm² so as to eliminate an aqueous stream with a flow rate of 0.23 t/h depleted in NaOH and to recover a stream of 4.44 t/h of alkaline aqueous phase enriched in catalyst (NaOH) and recycled as is to the feed of the reaction step. The electrodialysis apparatus consists of 34 cells providing an exchange surface area of 13.3 m². The efficiency of the electrodialysis apparatus is monitored by measuring the conductivity of the various streams of soda-containing aqueous phase: around 140 mS/cm for the concentrated alkaline aqueous phase (2.9% NaOH) and 16 mS/cm for the diluted alkaline aqueous phase (0.3% NaOH).

The composition by weight of the catalyst-depleted aqueous stream is 0.05% acetone, 0.85% isophorone, 0.3% NaOH and 98.8% water. This stream is directed to a wastewater treatment S_{E}.

The concentrations by weight of NaOH and of isophorone in the catalyst-enriched aqueous solution are 2.9% and 0.85%, respectively.

With such recycling of the catalyst, the consumption at the feed of the reaction step of the isophorone synthesis process is limited to 0.95 kg of NaOH (corresponding to 0.7 kg removed to S_{E} at the outlet of the electrodialysis apparatus and to 0.25 kg of NaOH dissolved in the crude isophorone stream) and to 2 litres of water per tonne of pure isophorone produced. The loss of isophorone contained in the aqueous stream and removed to the wastewater treatment is 2 kg per tonne of pure isophorone.

The invention therefore makes it possible to save:
130 kg of sodium hydroxide, 38 kg of isophorone and 4.3 m³ of water per tonne of isophorone, compared to a process without any recycling of the aqueous phase;
6 kg of sodium hydroxide and 14 litres of water per tonne of isophorone, compared to a process with recycling via a simple purge of the aqueous phase.

The invention thus makes it possible to recycle 99.2% of the catalyst used in the isophorone synthesis process and 95% of the isophorone contained in the alkaline aqueous phase at the outlet of the hydrolysis column.

In addition, this process makes it possible to save an amount of energy of 129 kWh compared to a process that eliminates excess water (~230 kg) by a thermal evaporation process which would require 135 kWh; the electrodialysis consuming only 6 kWh per tonne of isophorone.

More generally, the quantity I′ water to be removed in the process according to the invention is between 200 and 250 kg per tonne of isophorone, depending on the amount of by-products formed, the degree of hydrolysis and the amount of water entrained with the crude isophorone. The concentration by weight of water in the stream S_{E} is greater than 98.5%, in addition to the residual catalyst, the balance essentially corresponds to isophorone and traces of acetone.

### Balance of Example 3 Over the Whole Process: From Feed to the Final Isophorone

A stream of acetone with a flow rate of 1.4 t/h and a stream of an aqueous solution containing 30% of catalyst and 70% of water with a flow rate of 0.003 t/h are introduced into the reactor. The reactor is also fed by two recycle streams, defined below.

The flow rate of the stream of the reaction medium leaving the reactor is 14.8 t/h.

The flow rate of the stream of the organic phase leaving the reactive column is 1.28 t/h. This organic phase contains more than 79% isophorone.

The flow rate of the stream of the purified isophorone fraction leaving the distillation column is 1 t/h.

The flow rate of the stream of the light fraction from the reactive column, which recycles this fraction to the reactor, is 9 t/h.

The flow rate of the stream of the aqueous phase leaving the reactive column and decanter is 4.67 t/h. This aqueous phase contains 2.8% catalyst, 0.9% organic compounds, the remainder being water. This aqueous phase is introduced into the electrodialysis apparatus.

At the outlet of the electrodialysis apparatus, the flow rate of the stream of the catalyst-depleted phase is 0.23 t/h. The catalyst-depleted phase contains 0.9% organic compounds, 0.3% catalyst, the remainder being water. The flow rate of the stream of catalyst-enriched phase recycled to the feed of the reactor is 4.44 t/h. The catalyst-enriched phase contains 2.9% catalyst, 0.9% organic compounds, the remainder being water.

Thus, the use of the electrodialysis apparatus and the recycling of the catalyst-enriched phase allows a reduced consumption of water and catalyst (0.003 t/h), a minimized volume of aqueous effluent (0.23 t/h), a catalyst content in the effluent that is divided by 10 and a reduced energy consumption.

1. Process, preferably a continuous process, for the liquid-phase synthesis of isophorone by alkaline self-condensation of acetone, comprising the following successive steps:
a) condensation reaction of acetone within a reactor in an alkaline medium, followed by
b) distillation, optionally reactive distillation, of the reaction mixture obtained from the reactor, followed by
c) separation of the stream recovered at the bottom of the distillation column, optionally reactive distillation column, of step b) so as to separate the alkaline aqueous phase from the organic phase, followed by
d) extraction and/or purification of the organic phase to recover isophorone,
characterized in that the method comprises the following successive steps:
e) continuous or batch electrodialysis treatment of the alkaline aqueous phase recovered at the end of step c)
f) recycling to the reactor in step a) of the aqueous phase obtained from the electrodialysis which has an alkali metal hydroxide content greater than the alkali metal hydroxide content of the aqueous phase recovered at the end of step c).

2. Process according to claim 1, characterized in that the electrodialysis apparatus comprises at least one ion-exchange membrane comprising a polymer-based matrix comprising at least one fluorinated polymer or copolymer, preferably PVDF.

3. Process according to claim 1, characterized in that the electrodialysis apparatus has a total active exchange surface area of between 1 and 10 m² per tonne of alkaline aqueous phase to be treated, and preferably of between 2 and 5 m² per tonne of alkaline aqueous phase to be treated.

4. Process according to claim 1, characterized in that a current density of between 20 and 200 mA/cm², preferably of between 30 and 100 mA/cm², is applied to the electrodialysis apparatus.

5. Process according to claim 1, characterized in that the electrodialysis apparatus comprises several electrodialysis units in parallel or in series.

6. Process according to claim 1, characterized in that the aqueous alkali metal hydroxide solution is an aqueous solution of sodium hydroxide or potassium hydroxide.

7. Process according to claim 1, characterized in that the concentration of alkali metal hydroxide in the aqueous phase present within the reactor is greater than or equal to 50 g/l, preferably between 50 and 200 g/l, preferably between 80 and 150 g/l and more preferentially between 100 and 150 g/l.

8. Process according to claim 1, characterized in that the ratio of the mass flow rate of the stream of aqueous alkali metal hydroxide solution supplied (Qalkali metal hydroxide) to the mass flow rate of the organic stream supplied (Qorga) is between 0.25 and 1.0, preferably between 0.4 and 0.8 and more preferentially between 0.5 and 0.7.

9. Process according to claim 1, characterized in that the concentration of alkali metal hydroxide in the aqueous solution at the feed zone is between 5 and 40 g/l, preferably between 10 and 40 g/l and more preferentially between 15 and 35 g/l.

10. Use of an electrodialysis apparatus as defined in claim 1 for treating at least one alkaline aqueous effluent obtained from a process for the synthesis of isophorone by alkaline self-condensation of acetone in the liquid phase.
